(19) United States
(12) Patent Application Publication
(10) Pub. No.: US 2010/0137357 A1
(43) Pub. Date: Jun. 3, 2010

(54) COMPOSITIONS AND METHODS FOR HYPERHIDROSIS

(76) Inventors: John J. Koleng, Austin, TX (US); Josef Rossart, Austin, TX (US)

Correspondence Address: King & Spalding LLP, 401 Congress Avenue, Suite 3200, Austin, TX, 78701, US

(21) Appl. No.: 12/625,177

(22) Filed: Nov. 24, 2009

Related U.S. Application Data
(60) Provisional application No. 61/200,258, filed on Nov. 26, 2008.

(51) Int. Cl. A61K 31/46; A61P 43/00

(52) U.S. Cl. 514/304

(57) ABSTRACT

The present disclosure relates, according to some embodiments, to compositions, systems, and methods for ameliorating, preventing, and/or treating (collectively “treating”) hyperhidrosis and/or excessive perspiration (collectively “hyperhidrosis”). For example, a method for treating hyperhidrosis may comprise administering (e.g., topically administering) a composition comprising an anticholinergic compound (e.g., an ipratropium compound) to a mammal. A composition (e.g., a topical composition) for treating hyperhidrosis may comprise an anticholinergic compound (e.g., an ipratropium compound) and a vehicle (e.g., a physiologically acceptable vehicle). In some embodiments, administering a composition comprising an anticholinergic compound may comprise contacting the subject with a medicated article comprising the composition.

### CROSS-REFERENCE TO RELATED APPLICATIONS

This application claims the benefit of U.S. Provisional Application No. 61/200,258 filed Nov. 26, 2008, the entire contents of which are hereby incorporated in their entirety by reference.

### FIELD OF THE DISCLOSURE

The present disclosure relates, in some embodiments, to compositions, systems, and methods for ameliorating, preventing, and/or treating hyperhidrosis and/or excessive perspiration.

### BACKGROUND OF THE DISCLOSURE

Hyperhidrosis is an idiopathic pathological condition characterized by excessive, uncontrollable sweating beyond that required to cool the body. Disturbance of the central nervous system has been associated with this condition. Hyperhidrosis affects approximately 3% of the population. Hyperhidrosis not only may result in intense social embarrassment, but also may interfere with a person's occupation and safety.

Hyperhidrosis may have an effect on one or more body areas, especially the hands, axillae, feet, face; and/or the whole body. Palmar hyperhidrosis is the most common form. There are two types of hyperhidrosis: primary focal and secondary generalized. Primary focal hyperhidrosis refers to excessive sweating that is not caused by another medical condition, nor is it a side effect of medications. Excessive sweating itself is the medical condition and disease presentation. This type of sweating occurs on very specific areas of the body (described as focal) and is usually relatively “symmetric”-meaning that both the left and right sides of the body are affected similarly. The most common focal areas are the hands, feet, underarms, and head or face.

The other main type of hyperhidrosis is referred to as secondary generalized hyperhidrosis. This type of excessive sweating results from a medical condition other than primary hyperhidrosis (e.g. menopause, cancer, or diabetes) or a side effect of a medication. People with secondary hyperhidrosis experience sweating on larger areas of the body or on areas other than or in addition to the hands, feet, underarms, and head or face.

Antiperspirants (e.g., antiperspirants comprising aluminum) may be ineffective in treating these forms of uncontrolled, excessive perspiration. Oral medications may occasionally be effective, but they may have side effects that outweigh any benefits of treatment. Surgical procedures such as endoscopic thoracic sympathectomy may be used in the most severe cases. Although surgery affords permanent benefit in approximately 40% to 90% of subjects, it is invasive, requires general anesthesia, and is not without risk of undesirable side effects. Nearly 50% of these surgical patients will develop compensatory sweating of the trunk and/or thighs.

Botulium A neurotoxin (Botox), which blocks the action of acetylcholine released by the autonomic nerves on sweat glands, may be effective in the treatment of hyperhidrosis. Treatment with Botox involves the injection of small amounts of Botox into the affected areas resulting in a significant reduction in sweat production in the treated areas but on a temporary basis. Botox injections are effective at treating hyperhidrosis for several months, but repeat injections are necessary for continuous treatment and its safety has been questioned.

Topical glycopyrrolate may be used to treat gustatory sweating (not considered a form of hyperhidrosis) associated with diabetic autonomic neuropathy, although glycopyrrolate is not indicated for this condition. In this disorder, profuse sweating, starting on the forehead and then involving the face, scalp, and neck, begins soon after the subject ingests food. A glycopyrrolate solution may be applied to the face of a subject with gustatory sweating.

Similarly, glycopyrrolate maybe used to treat gustatory sweating associated with Frey's syndrome that may develop after parotidectomy. Frey's syndrome is believed to result from the aberrant reinnervation of the sweat glands of the face by the severed parotid parasympathetic nerve fibers. In both diabetic gustatory sweating and Frey's syndrome, profuse facial sweating is induced by the specific stimulus of eating. Moreover, sweating in each is a consequence of a distinct neuropathological process. In contrast, hyperhidrosis occurs spontaneously with or without the necessity or presence of a specific stimulus.

Furthermore, glycopyrrolate administered with iontophoresis may be used to treat hyperhidrosis although the topical use of glycopyrrolate is not a currently approved indication. This type of treatment required administration in the controlled setting of a physician's office, and it resulted in side effects such as abdominal discomfort in some patients. Other patients do not respond to this type of therapy.

### SUMMARY

Accordingly, a need has arisen for improved compositions, systems, and methods for ameliorating, preventing, and/or treating hyperhidrosis and/or excessive perspiration.

The present disclosure relates, according to some embodiments, to compositions, systems, and methods for ameliorating, preventing, and/or treating (collectively “treating”) hyperhidrosis and/or excessive perspiration (collectively “hyperhidrosis”). For example, a method for treating hyperhidrosis may comprise administering (e.g., topically administering) a composition comprising an anticholinergic compound (e.g., an ipratropium compound) to a mammal. A composition (e.g., a topical composition) for treating hyperhidrosis may comprise an anticholinergic compound (e.g., an ipratropium compound) and a vehicle (e.g., a physiologically acceptable vehicle).

The present disclosure relates, according to some embodiments, to methods for ameliorating, preventing, and/or treating (collectively “treating”) hyperhidrosis and/or excessive perspiration (collectively “hyperhidrosis”). For example, a method may comprise topically administering to the subject a composition comprising an effective amount of a pharmaceutically acceptable ipratropium compound, wherein hyperhidrosis and/or excessive perspiration is ameliorated, prevented, and/or treated. A subject may be a mammal (e.g., a mammal having one or more sweat glands) in some embodiments. Examples of mammal may include, without limitation, non-human primates, equine animals (e.g., horses), and humans. According to some embodiments, topically administering may comprise topically administering to the human's hand, axillae, groin, feet, face, neck, or combinations thereof. In some embodiments, neither the method as a whole nor topically administering in particular includes systemically distributing the ipratropium compound. A method may comprise, according to some embodiments, identifying a subject susceptible to hyperhidrosis and/or excessive perspiration.

The present disclosure relates, according to some embodiments, to compositions for ameliorating, preventing, and/or treating (collectively “treating”) hyperhidrosis and/or excessive perspiration (collectively “hyperhidrosis”). For example, a composition may comprise an ipratropium compound. An ipratropium compound may comprise, in some embodiments, a material selected from the group consisting of an ipratropium salt (e.g., a halide salt), an ipratropium analogue, an ipratropium derivative, an ipratropium prodrug, an ipratropium hydrate, an ipratropium isomer, or an ipratropium metabolite. According to some embodiments, an ipratropium compound may have the following formula:

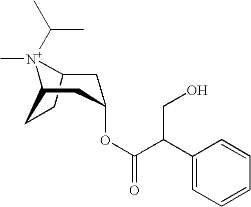

and may be optionally complexed with a pharmaceutically acceptable counter ion salt. A composition may comprise from about 0.25% (w/w) to about 25% (w/w) ipratropium compound in some embodiments. An ipratropium salt may comprise, according to some embodiments, a bromide salt, a chloride salt, an iodide salt, and/or combinations thereof. A composition may be formulated as a dosage form selected from a liquid, a syrup, a paste, a cream, a lotion, an ointment, a gel, a stick, a roll-on, a spray, a foam, a mousse, and combinations thereof in some embodiments. Examples of a liquid dosage form may include, without limitation, an aqueous solution, an alcoholic solution, and combinations thereof. According to some embodiments, a composition may comprise a pharmaceutically acceptable excipient.

The present disclosure also relates, according to some embodiments, to methods for ameliorating, preventing, and/or treating (collectively “treating”) hyperhidrosis and/or excessive perspiration (collectively “hyperhidrosis”) in a subject. For example, a method may comprise administering to a subject (e.g., a human subject) a dosage form comprising an effective amount of a pharmaceutically acceptable ipratropium compound, wherein hyperhidrosis and/or excessive perspiration is ameliorated, prevented, and/or treated and the dosage form is selected from the group consisting of a syrup, a paste, a cream, a lotion, an ointment, a gel, a stick, a roll-on, a spray, and combinations thereof. According to some embodiments, administering may comprise topically administering to the subject's hand, axillae, groin, feet, face, neck, or combinations thereof. An ipratropium compound may comprise, in some embodiments, a material selected from the group consisting of an ipratropium salt (e.g., a halide salt), an ipratropium analogue, an ipratropium derivative, an ipratropium prodrug, an ipratropium hydrate, an ipratropium isomer, or an ipratropium metabolite.

The present disclosure also relates, according to some embodiments, to methods for reducing perspiration in a subject. For example, a method may comprise topically administering to the human a composition comprising an effective amount of a physiologically acceptable ipratropium compound, wherein perspiration is reduced. In some embodiments, topically administering may comprise contacting the human with a medicated article comprising the composition comprising an effective amount of a pharmaceutically acceptable ipratropium compound. An article (e.g., an applicator) may include, in some embodiments, a peel, a pad, a patch, a bandage, a gel (e.g., a hydrogel), or a wrap. A composition may be coated on an article (e.g., an article surface) and/or dispersed within an article according to some embodiments.

### BRIEF DESCRIPTION OF THE DRAWINGS

Some embodiments of the disclosure may be understood by referring, in part, to the present disclosure and the accompanying drawings, wherein:

FIG. 1A illustrates the perspiration-blocking effects of a composition according to a specific example embodiment of the disclosure when applied to subject number 1 prior to exercise (12 hours and 2 hours beforehand);

FIG. 1B illustrates the perspiration-blocking effects of a composition according to a specific example embodiment of the disclosure when applied to subject number 2 prior to exercise (12 hours and 2 hours beforehand);

FIG. 2A illustrates the perspiration-blocking effects of a composition according to a specific example embodiment of the disclosure when applied to subject number 1 prior to exercise (2 hours and half an hour beforehand); and

FIG. 2B illustrates the perspiration-blocking effects of a composition according to a specific example embodiment of the disclosure when applied to subject number 2 prior to exercise (2 hours and half an hour beforehand).

### DETAILED DESCRIPTION

The present disclosure relates, in some embodiments, to compositions, systems, and methods for reducing perspiration in a mammal including, for example, ameliorating, preventing, and/or treating hyperhidrosis and/or excessive sweating in a mammal.

### Compositions

According to some embodiments, a compound may have the formula:

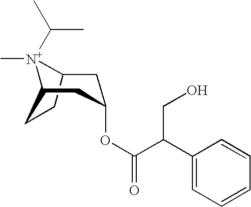

and optionally may be in salt form and/or complexed with a pharmaceutically acceptable counter ion salt. An ipratropium compound may include isomers, analogues and/or derivatives of ipratropium that are capable of inhibiting hyperhidrosis and/or excessive sweating. For example, the chemical structure may be modified so as to introduce, modify, and/or remove one or more functionalities of the structure. For example, such modification may result in the removal of an —OH functionality, the introduction of an amine functionality, the introduction of a halo functionality, and the like. According to some embodiments, ipratropium analogues and isomers may include any ipratropium analogue or isomer capable of inhibiting hyperhidrosis and/or excessive sweating. Compositions or preparations, in some embodiments, may include pharmaceutically acceptable excipients as desired to optimize the formulations, preferably topical formulations.

Ipratropium is an anticholinergic (parasympatholytic) agent which, based on animal studies, appears to inhibit vagally-mediated reflexes by antagonizing the action of acetylcholine, the transmitter agent released at the neuromuscular junctions. In humans, ipratropium bromide, an inorganic salt of ipratropium, has antisecretory properties and, when applied locally to the nasal mucosa, inhibits secretions from the serous and seromucous glands. Ipratropium is a quaternary amine that minimally crosses the nasal and gastrointestinal membranes and the blood-brain barrier, resulting in a reduction of the systemic anticholinergic effects (e.g., neurologic, ophthalmic, cardiovascular, and gastrointestinal effects) that are seen with tertiary anticholinergic amines. It is poorly absorbed into the systemic circulation following oral administration. Ipratropium, and more particularly, ipratropium bromide, may be administered via nasal spray for the treatment of rhinorrhea or by inhalation for the treatment of obstructive lung diseases; conditions for which ipratropium is approved in the U.S. Ipratropium is a non-selective muscarinic antagonist. It is a derivative of atropine, but is a quaternary amine, and therefore does not substantially cross the blood-brain barrier.

Ipratropium compounds, for use in the treatment of hyperhidrosis, may be effective over a wide dosage range and may be administered in a therapeutically effective amount. Preferably the topically administered composition contains about 0.25% to about 25% by weight of an ipratropium compound. It will be understood, however, that the amount of the ipratropium compound actually administered may be determined by a medical doctor in light of relevant circumstances, including: the condition to be treated; the chosen route of administration; the actual compound administered and its relative activity; the age, weight, and response of the individual subject; the severity of the subject's symptoms, and the like.

A pharmaceutically acceptable counter salt may include salts that retain the biological effectiveness and properties of the ipratropium compounds of this disclosure, that are not biologically or pharmaceutically unacceptable, and/or which carry an anionic charge according to some embodiments. The ipratropium compounds of this disclosure form salts by virtue of the presence of the quaternary ammonium thereon. An ipratropium composition (e.g., topical application) may comprise ipratropium, or an ipratropium compound such as an ipratropium salt, an ipratropium analogue, an ipratropium derivative, an ipratropium prodrug, an ipratropium hydrate, an ipratropium isomer, or an ipratropium metabolite according to some embodiments.

In some embodiments a composition may comprise pharmaceutically acceptable salt form of ipratropium. For example, a composition may comprise a pharmaceutically acceptable counter salt. Pharmaceutically acceptable salts of ipratropium may comprise, in some embodiments, an ipratropium cation and an anion of a mono or polyvalent acid (e.g., a hydrohalic acid). A salt form may include, without limitation, a halide salt (e.g., bromide salt, a chloride salt, and an iodide salt). According to some embodiments, a composition may comprise an ipratropium salt, solvate, ester or isomer. Pharmaceutically acceptable counter salts may be prepared from inorganic and organic acids. Salts derived from inorganic acids include, without limitation hydrochloric acid, hydrotropic acid, hydrogen fluoride, hydrogen iodide, sulfuric acid, nitric acid, phosphoric acid, and combinations thereof. Salts derived from organic acids may include without limitations acetic acid, propionic acid, glycolic acid, pyruvic acid, oxalic acid, maleic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and combinations thereof.

According to some embodiments, a product composition comprising an ipratropium compound may be administered in combination with an acceptable carrier adapted for topical administration. Topical compositions may be in the form of a solution, cream, ointment, mousse, gel, lotion, powder, patch, or aerosol formulation adapted for application to the skin. A composition (e.g., a topical composition) may comprise an ipratropium compound at a concentration from about 0.25% to about 25% by weight of the active compound in admixture with acceptable excipients in some embodiments. For example, a composition may comprise an ipratropium compound at a concentration of from about 0.25% (w/w) to about 2.5% (w/w), from about 2.5% (w/w) to about 10% (w/w), from about 10% (w/w) to about 20% (w/w), from about 20% (w/w) to about 25% (w/w), from about 0.25% (w/w) to about 1% (w/w), from about 1% (w/w) to about 8% (w/w), from about 8% (w/w) to about 15% (w/w), and/or from about 15% (w/w) to about 25% (w/w),

In some embodiments, an acceptable excipient may include carrier materials and/or other ingredients commonly used in the pharmaceutical and/or cosmetic arts. A composition may retain the biological effectiveness and properties of the ipratropium compounds and are not biologically or pharmaceutically unacceptable. Examples of acceptable excipients include without limitation alcohols, aloe vera gel, allantion, glycerin, vitamin A and E oils, mineral oil, PPG2, myristyl propionate, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, methyl cellulose, and combinations thereof. Lubrication agents such as talc, magnesium stearate, mineral oil, and stearic acid; wetting agents; emulsifying and suspending agents; preserving agents such as methyl- and propylhydroxy-benzoates; sweetening agents; and flavoring agents may also be included in the formulations. Colorants, fragrances, and penetration enhancers may be used in the formulation of a drug product containing an ipratropium compound. A composition, in some embodiments may be formulated so as to provide immediate, sustained, and/or delayed release of an ipratropium compound after administration. According to some embodiments, a treated area may be occluded by the use of a bandage or other suitable material to facilitate administration.

An ipratropium composition, in some embodiments, may additionally include one or more optional excipients to achieve a required or desired effect. Each additive may be compatible with the ipratropium compound. Compatible additives include additives that do not comprise the safety, efficiency, and/or use of an ipratropium compound in the manner described herein.

Topical preparations containing an ipratropium compound may be admixed with a variety of carrier materials or acceptable excipients. When an excipient serves as a diluent, it may be a solid, semi-solid, or liquid, that acts as a vehicle, carrier and/or medium for the active ingredient. In some embodiments, a composition may be in the form (e.g., a dosage form) of a powder, a suspension, a macro-emulsion, a micro-emulsion, a solution, a syrup, an alcoholic solution, an anhydrous solution, an ointment, a topical cleanser, a cleansing cream, a skin gel, a skin lotion, a mousse, a roll-on, a semi-solid stick, a plaster, an aerosol or non-aerosol spray in cream or gel formulation, and a soft gelatin capsule for topical administration, and/or combinations thereof.

A composition, in some embodiments, may be administered in the form of a liposome delivery system, including, for example, small unilamellar vesicles, large unilamellar vesicles, and/or multilamellar vesicles. Liposomes may be formed from a variety of phospholipids, including, for example, cholesterol, stearylamine, and/or phosphatidylcholines. Other topical delivery systems such as microsponges may be used to topically administer ipratropium compounds.

A composition may have any suitable pH (e.g., any physiologically acceptable pH) in some embodiments. For example, a pH may be selected (e.g., tuned) such that it is the same as and/or compatible with the pH of the skin of the subject to receive the composition (e.g., to minimize irritation) and/or selected (e.g., tuned) to increase and/or maximize the stability of one or more components.

### Systems

The present disclosure relates, in some embodiments, to systems for reducing perspiration including, for example, ameliorating, preventing, and/or treating hyperhidrosis and/or excessive perspiration. For example, a system may include an article configured to contact a subject's skin and comprising an ipratropium compound. An article (e.g., an applicator) may include, in some embodiments, a peel, a pad, a patch, a bandage, or a wrap. For example, it may be desirable and/or required to maintain aseptic conditions at and/or near a wound (e.g., a surgical incision, a puncture wound, an abrasion). To reduce or prevent sweating that may otherwise threaten to compromise these aseptic conditions, a wound dressing comprising a composition including an ipratropium compound may be applied. A chemical (e.g., a coating) or mechanical (e.g., a covering) may be included, in some embodiments, to limit and/or prevent unintentional transference of a ipratropium composition to another subject (e.g., body to body transfer).

In preparing a formulation for topical administration, an active compound may be milled to provide the appropriate particle size prior to combining with the other excipients. The need and degree of milling will depend, at least in part, upon the solubility of the ipratropium compound and the desired aesthetic properties (e.g., smoothness, texture) of the final product.

### Methods of Administration

The present disclosure relates, in some embodiments, to methods for ameliorating, preventing, and/or treating perspiration in a subject. For example, a method may include administering a composition comprising an ipratropium compound to a subject prior to or during activity and/or stress that may be associated with perspiration (e.g., a soldier, a laborer, an athlete). According to some embodiments, reduced perspiration may maintain and/or improve performance. In some embodiments, a method may include administering a composition comprising an ipratropium compound to a subject having and/or at risk of having hyperhidrosis, which includes a condition commonly known and understood in the art and/or known and understood by an average consumer lacking any medical training. It may be an idiopathic condition characterized by excessive, uncontrollable perspiration beyond that required to cool the body. Hyperhidrosis may also include excessive perspiration in response to the body's reaction to cool itself or as an anxiety response according to some embodiments.

According to some embodiments, an ipratropium compound may be administered topically to a subject (e.g., a mammal), A mammal may include, in some embodiments, a mammal having one or more sweat glands (e.g., to regulate body temperature). According to some embodiments, a mammal may include a mouse, a rat, an ovine animal, a bovine animal, an equine animal, a non-human primate, and/or a human. A dose (e.g., a daily dose) of an ipratropium compound may vary depending on the medical condition of the subject, the severity of the condition, the skin status, the age of the subject, the degree of perspiration reduction desired and/or required (e.g, to facilitate an activity) and/or conditions under which symptoms present themselves. Ipratropium compounds, in some embodiments may be administered from once to multiple times daily or weekly in single or divided multiple doses. An ipratropium compound may be applied to the body of a subject including the hands, face, scalp, neck, trunk, back, limbs, axillae, scalp, groin, legs, and/or feet of the mammal. An ipratropium compound may be administered without the concurrent use or subsequent administration of iontophoresis to the skin according to some embodiments.

Subjects susceptible to hyperhidrosis and/or excessive perspiration include, but are not limited to, subjects exhibiting idiopathic post stroke, central nervous system disease, and/or injury resulting in these conditions subjects with hyperhidrosis may include individuals that excessively perspire while engaging in physical activity or are induced to excessively sweat due to unfavorable or harsh environmental conditions (e.g., hot and/or humid environments, wearing of uniforms or protective gear). Subjects with hyperhidrosis may include individuals that excessively perspire under psychological distress, e.g. stressful, embarrassing, fearful or anxious situations causing anxiety, fear and/or nervousness.

An area of skin treated with an ipratropium composition may perspire less than a corresponding untreated area of skin, according to some embodiments. For example, a treated area may display a reduction in perspiration over a corresponding untreated area of up to about a 5% reduction, up to about a 10% reduction, up to about a 15% reduction, up to about a 20% reduction, up to about a 25% reduction, up to about a 30% reduction, up to about a 35% reduction, up to about a 40% reduction, and/or over about a 40% reduction.

In some embodiments, a composition may be administered as a liquid (e.g., an aqueous solution, an alcoholic solution), a syrup, a paste, a cream, a lotion, an ointment, a gel, a stick, a roll-on, a spray, or any other form. A subject's skin may be contacted with a composition directly (e.g., solution applied directly to skin) or included in an applicator that contacts skin.

According to some embodiments, a composition may be applied to a subject in a way that avoids systemic distribution of the ipratropium compound. Taking care to minimize and/or avoid systemic distribution may limit and/or prevent the occurrence of side effects associated with systemic distribution.

A therapeutically effective amount (e.g., a therapeutically effective amount of an ipratropium compound) may include, in some embodiments, an amount that will ameliorate, prevent, and/or treat hyperhidrosis and/or excessive perspiration. An effective amount may be the amount necessary to reduce, control, or alleviate excessive perspiration and may include an amount needed to achieve a rate or amount of perspiration considered normal to serve to cool the body.

As will be understood by those skilled in the art who have the benefit of the instant disclosure, other equivalent or alternative compositions, devices, methods, and systems for ameliorating, preventing, and/or treating hyperhidrosis and/or excessive perspiration can be envisioned without departing from the description contained herein. Accordingly, the manner of carrying out the disclosure as shown and described is to be construed as illustrative only.

Persons skilled in the art may make various changes in the shape, size, number, and/or arrangement of parts without departing from the scope of the instant disclosure. For example, the number and types of articles, if any, to contact skin for delivery of an anti-perspiration molecule may be varied. In some embodiments, a disposable article may be included as part of a reusable device for placing the disposable article in contact with skin. In addition, the size of an article, device and/or system may be scaled up (e.g., to be used for adult subjects) or down (e.g., to be used for juvenile subjects) to suit the needs and/or desires of a practitioner. Also, where ranges have been provided, the disclosed endpoints may be treated as exact and/or approximations as desired or demanded by the particular embodiment. Where the endpoints are approximate, the degree of flexibility may vary in proportion to the order of magnitude of the range. For example, a range of endpoint of about 50 may one the one hand include 50.5, but not 52.5 or 55 in the context of a range of about 5 to about 50 and, on the other hand, include 55, but not 60 or 75 in the context of a range of about 0.5 to about 50. In addition, it may be desirable, in some embodiments, to mix and match range endpoints. Also, in some embodiments, each figure disclosed (e.g., in one or more of the Examples and/or Drawings) may form the basis of a range (e.g., the disclosed value +/−about 10%, +/−about 100%) and/or a range endpoint. Persons skilled in the art may make various changes in methods of preparing and using a composition, device, and/or system of the disclosure. For example, a composition, device, and/or system may be prepared and or used as appropriate for animal and/or human use (e.g., with regard to sanitary, infectivity, safety, toxicity, biometric, and other considerations).

All or a portion of an article, device and/or system for reducing perspiration may be configured and arranged to be disposable, serviceable, interchangeable, and/or replaceable. These equivalents and alternatives along with obvious changes and modifications are intended to be included within the scope of the present disclosure. Accordingly, the foregoing disclosure is intended to be illustrative, but not limiting, of the scope of the disclosure as illustrated by the following claims.

### EXAMPLES

Some specific example embodiments of the disclosure may be illustrated by one or more of the examples provided herein.

### Example 1

### Ipratropium Alcohol Topical Solution

A specific example embodiment of an ipratropium composition may be prepared as follows:

| No. | Material Description | Batch Formula (g/100 g) |
|---|---|---|
| 1 | Ipratropium Bromide* | 2.59 |
| 2 | Ethanol 95% (v/v) | 32.47 |
| 3 | Purified Water | 69.94 |

| | |
|---|---|
| Total | 100.00 |

*As the monohydrate (ipratropium MW = 332.466; ipratropium bromide MW = 412.37; ipratropium bromide monohydrate MW = 430.4).

### Preparation:

Mix the required quantity of Ethanol (2) and Purified Water (3) in a suitable container. Add the required quantity of Ipratropium Bromide (1) to Ethanol/Water mixture. Mix. Package in suitable container.

The above formulation is suitable for application to the affected sites using a cotton pad or similar applicator. The solution may be poured onto applicator pads or wipes in a suitable container to provide for ‘ready to use’ pads or wipes.

### Example 2

### Ipratropium Ointment #1

A specific example embodiment of an ipratropium composition may be prepared as follows:

| No. | Material Description | Batch Formula (g/100 g) |
|---|---|---|
| 1 | Ipratropium Bromide* | 2.59 |
| 2 | White Wax | 4.88 |
| 3 | White Petrolatum | 92.53 |

| | |
|---|---|
| Total | 100.00 |

*As the monohydrate.

### Preparation:

Melt White Wax (2) in suitable container using a water bath.

Add White Petrolatum (3) and mix until uniform.

Add Ipratropium Bromide (1) and mix until uniform.

Remove from heat and continue to mix until cool.

Package in suitable container.

### Example 3

### Ipratropium Ointment #2

A specific example embodiment of an ipratropium composition may be prepared as follows:

| No. | Material Description | Batch Formula (g/100 g) |
|---|---|---|
| 1 | Ipratropium Bromide* | 2.59 |
| 2 | Cholesterol | 2.92 |
| 3 | Stearyl Alcohol | 2.92 |
| 4 | White Wax | 7.79 |
| 5 | White Petrolatum | 83.78 |

| | |
|---|---|
| Total | 100.00 |

*As the monohydrate.

### Preparation:

Combine Stearyl Alcohol (3), White Wax (4), and White Petrolatum (5) in suitable container and melt using a water bath.

Add Cholesterol (2) and mix until uniform.

Add Ipratropium Bromide (1) and mix until uniform.

Remove from heat and continue to mix until congealed.

Package in suitable container.

### Example 4

### Ipratropium Ointment #3

A specific example embodiment of an ipratropium composition may be prepared as follows:

| No. | Material Description | Batch Formula (g/100 g) |
|---|---|---|
| 1 | Ipratropium Bromide* | 2.590 |
| 2 | Methyparaben | 0.024 |
| 3 | Propylparaben | 0.015 |
| 4 | Sodium Lauryl Sulfate | 0.974 |
| 5 | Propylene Glycol | 11.689 |
| 6 | Stearyl Alcohol | 24.353 |
| 7 | White Petrolatum | 24.353 |
| 8 | Purified Water | 36.002 |

| | |
|---|---|
| Total | 100.00 |

*As the monohydrate.

### Preparation:

Combine Stearyl Alcohol (6) and White Petrolatum (7) in a suitable container and melt using a water bath to about 75° C.

Combine Ipratropium Bromide (1), Methylparaben (2), Propylparaben (3), Sodium Lauryl Sulfate (4), and Propylene Glycol (5) in the Purified Water (8) and mix.

Heat the mixture to 75° C.

Add the water mixture (B) to the Stearyl Alcohol/White Petrolatum mixture (A).

Remove the combined mixture from the heat; stir rapidly and continuously until the mixture has congealed.

Package in a suitable container.

### Example 5

### Ipratropium Ointment #4

A specific example embodiment of an ipratropium composition may be prepared as follows:

| | | Batch |
|---|---|---|
| No. | Material Description | Formula (g/100 g) |
| 1 | Ipratropium Bromide* | 2.59 |
| 2 | Polyethylene Glycol 400 | 58.45 |
| 3 | Polyethylene Glycol 3350 | 38.96 |

| | |
|---|---|
| Total | 100.00 |

*As the monohydrate.

### Preparation:

Combine Polyethylene Glycol 400 (2) and Polyethylene Glycol 3350 (3) in a suitable container and melt using a water bath to 65° C.

Add Ipratropium Bromide (1) and mix until uniform.

Remove from heat and continue to mix until the preparation has congealed.

Package in a suitable container.

### Example 6

### Ipratropium Ointment #5

A specific example embodiment of an ipratropium composition may be prepared as follows:

| | | Batch |
|---|---|---|
| No. | Material Description | Formula (g/100 g) |
| 1 | Ipratropium Bromide* | 2.59 |
| 2 | Propylene Glycol | 2.43 |
| 3 | Triacetin | 2.43 |
| 4 | Mineral Oil | 55.50 |
| 5 | Microcrystalline Wax | 34.08 |
| 6 | Propylene Glycol Stearate | 2.92 |
| 7 | Citric Acid | 0.05 |

| | |
|---|---|
| Total | 100.00 |

*As the monohydrate.

### Preparation:

Combine Mineral Oil (4), Microcrystalline Wax (5), and Propylene Glycol Stearate (6) in a suitable container and melt at 75° C. to 85° C. Mix.

Combine Citric Acid (7), Ipratropium Bromide (1), Propylene Glycol (2) and Triacetin (3), Mix, use heat if necessary.

Allow Mineral Oil mixture (A) to cool to about 55° C. then add Ipratropium mixture while stirring to disperse finely and uniformly.

Continue to mix while cooling the ointment to 30° C. or lower.

Package in a suitable container.

### Example 7

### Ipratropium Stick #1

A specific example embodiment of an ipratropium composition may be prepared as follows:

| | | Batch |
|---|---|---|
| No. | Material Description | Formula (g/100 g) |
| 1 | Ipratropium Bromide* | 2.59 |
| 2 | Talc | 19.69 |
| 3 | Petrolatum | 20.73 |
| 4 | Paraffin Wax | 31.09 |
| 5 | Cocoa Butter | 15.54 |
| 6 | Beeswax | 10.36 |

| | |
|---|---|
| Total | 100.00 |

*As the monohydrate.

### Preparation:

Combine and mix Ipratropium Bromide (1) and Talc (2) with Petrolatum (3).—Triturate until smooth.

Combine and melt Paraffin Wax (4), Cocoa Butter (5), and Beeswax (6) with the Petrolatum Mixture (A) using a water bath.

Mix thoroughly.

Pour heated mixture (B) into molds or suitable containers.

### Example 8

### Ipratropium Stick #2

A specific example embodiment of an ipratropium composition may be prepared as follows:

| | | Batch |
|---|---|---|
| No. | Material Description | Formula (g/100 g) |
| 1 | Ipratropium Bromide* | 2.59 |
| 2 | Glyceryl Monostearate | 19.48 |
| 3 | Span 80 | 1.95 |
| 4 | Oil-in-Water Emulsion | 75.98 |
| | Base (Dermabase) | |

| | |
|---|---|
| Total | 100.00 |

*As the monohydrate.

### Preparation:

Melt Glyceryl Monostearate (2) at 55° C. to 70° C. in a beaker using a water bath.

Add Span 80 (3) to melted Glyceryl Monostearate (A) and mix thoroughly.

Add Ipratropium Bromide (1) to Span 80/Glyceryl Monostearate mixture and mix thoroughly.

Heat the Oil-in-Water Emulsion Base (4) to about 60° C. and pour into the previous mixture (C).

Remove from heat and stir the complete mixture rapidly.

Pour the cooled mixture into a suitable container.

### Example 9

### Ipratropium Stick #3

A specific example embodiment of an ipratropium composition may be prepared as follows:

| | | Batch |
|---|---|---|
| No. | Material Description | Formula (g/100 g) |
| 1 | Ipratropium Bromide* | 2.59 |
| 2 | Sodium Stearate | 6.82 |
| 3 | Alcohol | 63.32 |
| 4 | Propylene Glycol | 24.35 |
| 5 | Cyclomethicone | 2.92 |

| | |
|---|---|
| Total | 100.00 |

*As the monohydrate.

### Preparation:

Melt Sodium Stearate (2).

Combine and mix the Ipratropium Bromide (1), Alcohol (3), Propylene Glycol (4) and Cyclomethicone (5).

Add the Alcohol mixture (B) to the melted Sodium Stearate (A).

Mix well, cool slightly, and pour into stick molds.

### Example 10

### Ipratropium Alcohol Gel

A specific example embodiment of an ipratropium composition may be prepared as follows:

| No. | Material Description | Amount (g) |
|---|---|---|
| 1 | Ipratropium Bromide* | 1.12 |
| 2 | Base, Alcohol Gel | 20.47 |

| | |
|---|---|
| Total | 21.6 |

*As the monohydrate.

### Preparation:

Dispense 20.47 g alcohol gel base (Spectrum, Cat. #A1137) into suitable container. Dispense 1.12 g ipratropium bromide monohydrate (Spectrum) into container and mix into base with stirring. Package in a closed glass container protected from light. Store as desired at 2°-8° C.

### Example 11

### Ipratropium Gel #1

A specific example embodiment of an ipratropium composition may be prepared as follows:

| | | Batch |
|---|---|---|
| No. | Material Description | Formula (g/100 g) |
| 1 | Ipratropium Bromide* | 2.59 |
| 2 | Poloxamer 407 | 25.00 |
| 3 | Potassium Sorbate | 0.20 |
| 4 | Purified Water | 72.21 |

| | |
|---|---|
| Total | 100.00 |

*As the monohydrate.

### Preparation:

Combine and mix the Ipratropium Bromide (1), Potassium Sorbate (3) and Purified Water (4) in an ice bath.

Add the Poloxamer 407 (2) to the mixture above and stir. Keep cold.

Mix well until the Poloxamer is dissolved.

Package in a suitable container. Mixture will thicken at room temperature.

### Example 12

### Ipratropium Gel #2

A specific example embodiment of an ipratropium composition may be prepared as follows:

| | | Batch |
|---|---|---|
| No. | Material Description | Formula (g/100 g) |
| 1 | Ipratropium Bromide* | 2.59 |
| 2 | Methylcellulose 1500 cps | 3.00 |
| 3 | Purified Water | 94.41 |

| | |
|---|---|
| Total | 100.00 |

*As the monohydrate.

### Preparation:

Add the Methylcellulose (2) to about 50 g of boiling Purified Water (3) and disperse well.

Combine the remaining Purified Water (3) and the Ipratropium Bromide (1) and mix well in an ice bath.

Add the cold Ipratropium Bromide mixture (B) to the hot Methylcellulose mixture (A).

Mix until uniform and thickened.

Package in a suitable container.

### Example 13

### Ipratropium Gel #3

A specific example embodiment of an ipratropium composition may be prepared as follows:

| | | Batch |
|---|---|---|
| No. | Material Description | Formula (g/100 g) |
| 1 | Ipratropium Bromide* | 2.59 |
| 2 | Hydroxypropylcellulose | 1.61 |
| 3 | Isopropyl Alcohol 70% | 90.46 |
| 4 | Propylene Glycol | 3.77 |
| 5 | Polysorbate 80 | 1.57 |

| | |
|---|---|
| Total | 100.00 |

*As the monohydrate.

### Preparation:

Add the Hydroxypropylcellulose (2) to the Isopropyl Alcohol (3) and mix until a clear gel results.

Make a paste with the Ipratropium Bromide (1), Propylene Glycol (4) and Polysorbate. (5).

Add the Ipratropium Bromide mixture (B) to the Hydroxypropylcellulose mixture (A) by geometric dilution.

Package in a suitable container.

### Example 14

### Ipratropium Cream #1

A specific example embodiment of an ipratropium composition may be prepared as follows:

| | | Batch |
|---|---|---|
| | | Formula |
| No. | Material Description | (g/100 g) |
| 1 | Ipratropium Bromide* | 2.59 |
| 2 | Trilaneth-4 Phosphate and | 17.90 |
| | Glyceryl Stearate and PEG-2 | |
| | Stearate | |
| 3 | Hydrogenated Palm/Kernel Oil | 1.99 |
| | PEG-6 Esters | |
| 4 | Mineral Oil | 7.96 |
| 5 | Sodium Methylparaben | 0.03 |
| 6 | Sorbic Acid | 0.07 |
| 7 | Purified Water | 64.26 |
| 8 | Menthol | 0.20 |
| 9 | Resorcinol | 0.03 |
| 10 | Ethoxydiglycol | 4.97 |

| | |
|---|---|
| Total | 100.00 |

*As the monohydrate.

### Preparation:

Dissolve Menthol (8) and Resorcinol (9) in Ethoxydiglycol (10) with stirring.

Combine Ipratropium Bromide (1), Trilaneth mixture (2), Palm/Kernal Oil Esters (3), Mineral Oil (4), Sodium Methylparaben (5), and Sorbic Acid (6) with the Purified Water (7). Mix and heat to 75° C.; allow to cool slowly with constant stirring.

When Ipratropium Bromide mixture reaches 35° C., add it to the Menthol mixture with stirring. Homogenize if necessary.

Package in a suitable container.

### Example 15

### Ipratropium Cream #2

A specific example embodiment of an ipratropium composition may be prepared as follows:

| | | Batch |
|---|---|---|
| No. | Material Description | Formula (g/100 g) |
| 1 | Ipratropium Bromide* | 2.59 |
| 2 | Cetylstearyl Alcohol | 17.90 |
| 3 | Cremophor A 6 | 1.99 |
| 4 | Cremophor A 25 | 7.96 |
| 5 | Liquid Paraffin | 0.03 |
| 6 | Methylparaben | 0.07 |
| 7 | Purified Water | 64.26 |
| 8 | Propylene Glycol | 0.20 |

| | |
|---|---|
| Total | 100.00 |

*As the monohydrate.

### Preparation:

Heat and stir a mixture of Cetylstearyl Alcohol (2), Cremophor A 6 (3), Cremophor A 25 (4) and Liquid Paraffin (5) to 80° C.

In a separate container, heat and stir a mixture of Ipratropium Bromide (1), Propylene

Glycol (8), and Purified Water (7) to 80° C.

Add Ipratropium Bromide mixture (B) to Cetylstearyl Alcohol mixture (A) with rigorous stirring.

Remove from heat and continue to stir to cool to room temperature.

Package in a suitable container.

### Example 16

### Ipratropium Cream #3

A specific example embodiment of an ipratropium composition may be prepared as follows:

| | | Batch |
|---|---|---|
| No. | Material Description | Formula (g/100 g) |
| 1 | Ipratropium Bromide* | 2.59 |
| 2 | Propylene Glycol | 3.90 |
| 3 | Liquid Paraffin | 9.75 |
| 4 | Cetostearyl Alcohol | 6.89 |
| 5 | Cetomacrogol 1000 | 3.90 |
| 6 | Isopropyl Myristate | 4.87 |
| 7 | Dibasic Sodium Phosphate | 0.47 |
| 8 | Citric Acid Monohydrate | 0.15 |
| 9 | Imidurea | 0.24 |
| 10 | Purified Water | 67.24 |

| | |
|---|---|
| Total | 100.00 |

*As the monohydrate.

### Preparation:

Melt Cetostearyl Alcohol (4) and Cetomacrogol 1000 (5) in a fat-melting vessel at 70° C. Add Liquid Paraffin (3) and Isopropyl Myristate (6) and mix well. Hold the temperature between 60° C. and 70° C.

Add Purified Water (10) to a separate manufacturing vessel and heat to 70° C. to 80° C. Dissolve Dibasic Sodium Phosphate (7), Citric Acid (8), Ipratropium Bromide (1), Propylene Glycol (2), and Imidurea (9) in Purified Water (B). Hold the temperature between 60° C. and 70° C.

Transfer the Cetostearyl Alcohol mixture (A) through a stainless steel filter to the heated Water mixture (C) while stirring at a temperature of 60° C. to 70° C. Mix and homogenize for 10 minutes under vacuum at 0.5 bar. Cool the cream to a temperature of 25° C. to 30° C. with continuous stirring.

Package in a suitable container.

### Example 17

### Ipratropium Cream #4

A specific example embodiment of an ipratropium composition may be prepared as follows:

| | | Batch |
|---|---|---|
| No. | Material Description | Formula (g/100 g) |
| 1 | Ipratropium Bromide* | 2.59 |
| 2 | Polawax | 14.61 |
| 3 | Oleyl Alcohol | 3.90 |
| 4 | PEG-75 Lanolin | 4.87 |
| 5 | Mineral Oil (70 cS) | 14.61 |
| 6 | Dimethicone | 7.31 |
| 7 | Purified Water | 52.11 |

| | |
|---|---|
| Total | 100.00 |

*As the monohydrate.

### Preparation:

Heat with stirring Polawax (2), Oleyl Alcohol (3), PEG-75 Lanolin (4), and Mineral Oil (5) in a vessel at 60° C. to 65° C.

Heat and mix Ipratropium Bromide (1), Dimethicone (6), and Purified Water (7) in a separate vessel at 60° C. to 65° C.

Add Ipratropium Bromide mixture to Polawax mixture while stirring. Stir to cool to 30° C. Package in a suitable container.

### Example 18

### Ipratropium Lotion

A specific example embodiment of an ipratropium composition may be prepared as follows:

| | | Batch |
|---|---|---|
| | | Formula |
| No. | Material Description | (g/100 g) |
| 1 | Ipratropium Bromide* | 2.59 |
| 2 | PEG-6 Stearate and Cetech- | 4.94 |
| | 20 and Steareth-20 (TEFOSE | |
| | 2000) | |
| 3 | Mineral Oil | 2.97 |
| 4 | Cetyl Alcohol | 1.98 |
| 5 | Sodium Methylparaben | 0.07 |
| 6 | Sorbic Acid | 0.03 |
| 7 | Purified Water | 87.42 |

| | |
|---|---|
| Total | 100.00 |

*As the monohydrate.

### Preparation:

Heat and stir TEFOSE 2000 (2), Mineral Oil (3), and Cetyl Alcohol (4) to 75° C.

Allow to cool with stirring.

In a separate container, stir a mixture of Ipratropium Bromide (1), Sodium Methylparaben (5), Sorbic Acid (6) and Purified Water (7).

Add Ipratropium Bromide mixture (B) to TEFOSE mixture (A) with rigorous stirring.

Continue to stir to cool to room temperature.

Package in a suitable container.

### Example 19

### Ipratropium Alcohol Topical Spray

A specific example embodiment of an ipratropium composition may be prepared as follows:

| | | Batch |
|---|---|---|
| No. | Material Description | Formula (g/100 g) |
| 1 | Ipratropium Bromide* | 2.59 |
| 2 | Povidone | 1.00 |
| 3 | Ethanol 95% (v/v) | 32.47 |
| 4 | Purified Water | 63.94 |

| | |
|---|---|
| Total | 100.00 |

*As the monohydrate.

### Preparation:

Mix the required quantity of Ethanol (3) and Purified Water (4) in a suitable container.

Add the required quantity of Povidone (2). Mix until dissolved.

Add the required quantity of Ipratropium Bromide (1) to Povidone (B) mixture. Mix.

Fill into suitable vials and attached a spray pump closure.

### Example 20

### Method of Treatment

According to a specific example embodiment, a method of treating a subject may include:

1. Clean an area of skin to be treated (e.g., hands, face, scalp, neck, trunk, back, limbs, axillae, legs, feet, and/or groin).

2. Dry (optionally, actively or passively).

3. Apply an aliquot of a composition comprising about 0.25% to about 25% by weight of an ipratropium compound to the skin surface. Sufficient amounts of ipratropium composition may be employed to cover the entire skin surface that exhibits excessive sweating with a layer of the ipratropium compound. If necessary or desired, excess ipratropium compound may be removed from the skin with a suitable wipe at the time of application.

It has been found that application of an ipratropium compound to the skin of mammals suffering from hyperhidrosis or excessive perspiration reduces perspiration at the location where the ipratropium compound is applied. After application, the ipratropium compound penetrates the skin and is associated with few side effects.

### Example 21

### In Vivo Effects of an Ipratropium Composition

A permanent marker was used to divide the backs of two healthy adult male volunteers into three approximately even, lengthwise sections (left, center, and right), each spanning from top to bottom. A composition comprising 4% ipratropium as described in Example 10 was used to coat the left section of each subject's back about 12 hours and again about an hour and a half before performing an exercise test. The right section was left untreated. Between the last ipratropium administration and the exercise test, the left and right sections of each subject were coated with a 10% povidone iodine solution and, after drying, liberally dusted with starch prior to the exercise test.

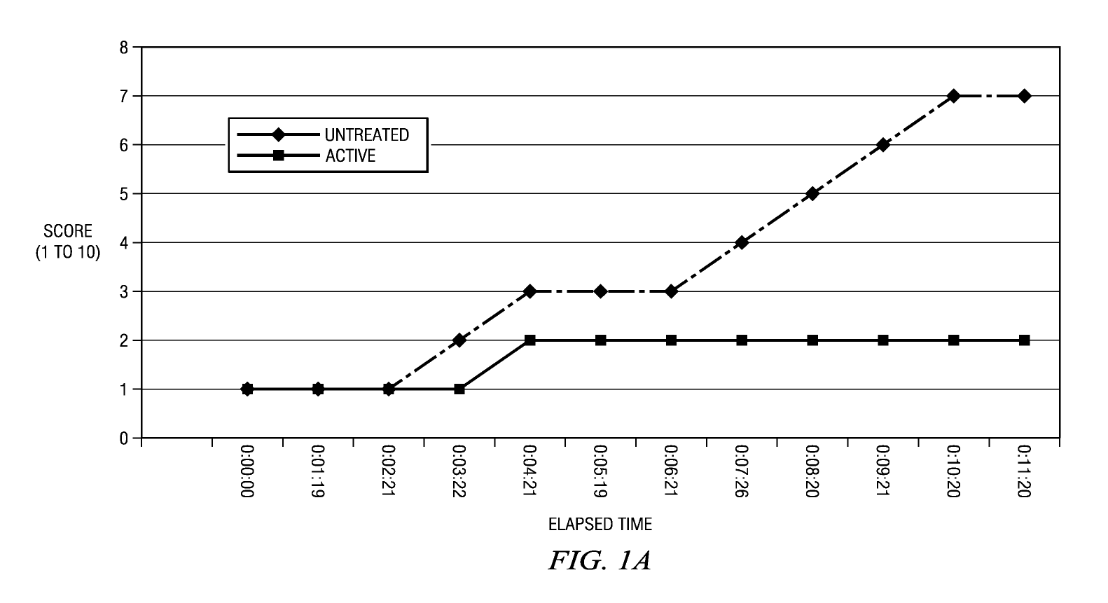

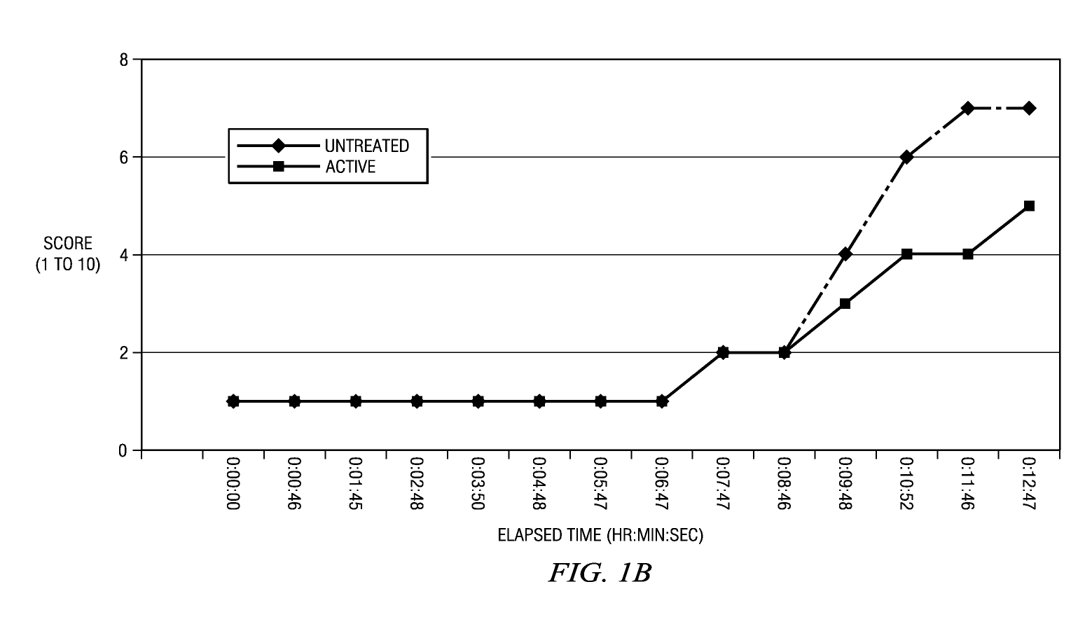

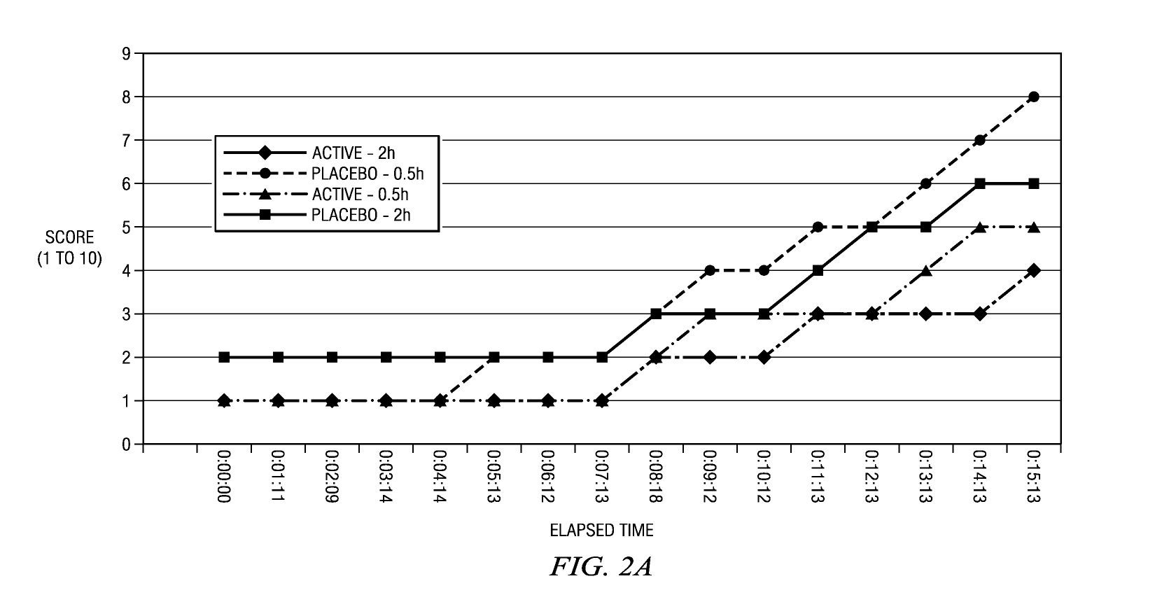

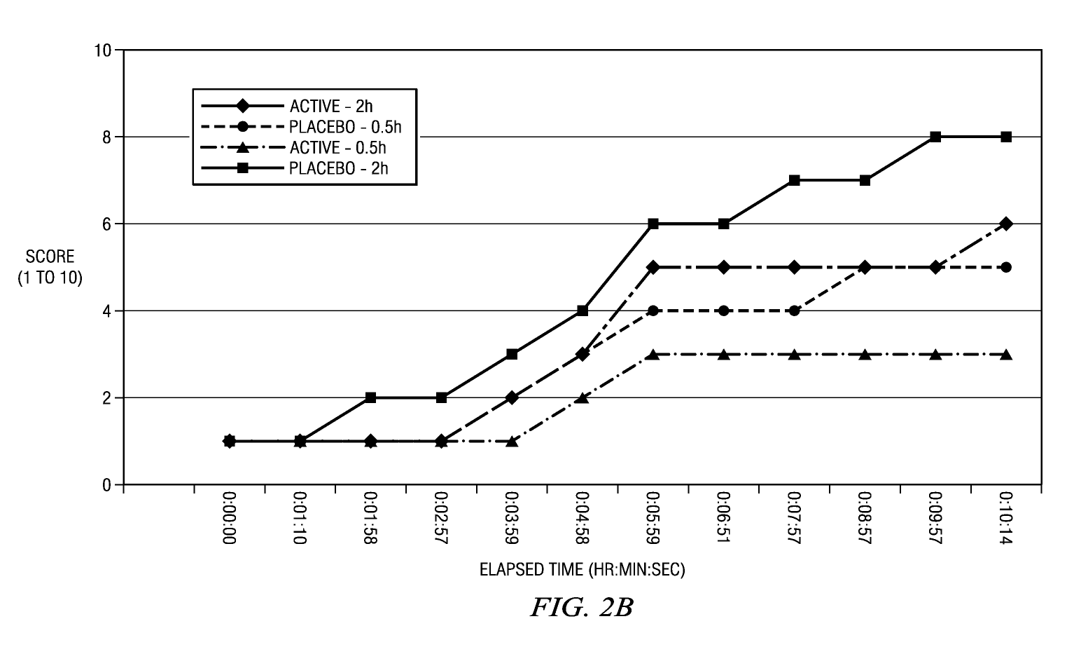

Exercise tests were performed by having each subject pedal a Lifecycle 6500 (random setting) for up to 15 minutes. Subjects remained shirtless during the test and their backs were photographed approximately once every minute. The degree of perspiration was assessed from the photographs using a 1-10 scale (1=no sweat and 10=severe sweating). Results are shown graphically for subjects 1 (FIG. 1A) and 2 (FIG. 1B).

Neither subject displayed any redness, irritation, or other adverse reactions indicating that the ipratropium composition was well-tolerated.

### Example 22

### In Vivo Effects of an Ipratropium Composition

A permanent marker was used to divide the backs of two healthy adult male volunteers into four approximately even, lengthwise quadrants (outer left, inner left, inner right, and outer right), each spanning from top to bottom. A composition comprising 4% ipratropium as described in Example 10 was used to coat (i) the outer left quadrant of each subject's back about 2 hours before and (ii) the inner right quadrant of each subject's back about half an hour before performing an exercise test. A composition comprising vehicle alone (Example 10 composition without ipratropium) was used to coat (i) the outer right quadrant of each subject's back about 2 hours before and (ii) the inner left quadrant of each subject's back about half an hour before the exercise test. Between the last ipratropium administration and the exercise test, the back of each subject was coated with a 10% povidone iodine solution and, after drying, liberally dusted with starch prior to the exercise test.

Exercise tests were performed by having each subject pedal a Lifecycle 6500 (random setting) for up to 15 minutes. Subjects remained shirtless during the test and their backs were photographed approximately once every minute. The degree of perspiration was assessed from the photographs using a 1-10 scale (1=no sweat and 10=severe sweating). Results are shown graphically for subjects 1 (FIG. 2A) and 2 (FIG. 2B).

Neither subject displayed any redness, irritation, or other adverse reactions indicating that the ipratropium composition was well-tolerated.

1. A method for ameliorating, preventing, and/or treating hyperhidrosis and/or excessive perspiration in a subject, the method comprising:
topically administering to the subject a composition comprising an effective amount of a pharmaceutically acceptable ipratropium compound, wherein hyperhidrosis and/or excessive perspiration is ameliorated, prevented, and/or treated.

2. A method according to claim 1, wherein the subject is a mammal.

3. A method according to claim 2, wherein the mammal is selected from the group consisting of a non-human primate, an equine animal and a human.

4. A method according to claim 2, wherein the mammal is a mammal having one or more sweat glands.

5. A method according to claim 1, wherein the subject is a human.

6. A method according to claim 5, wherein topically administering to the human further comprises topically administering to the human's hand, axillae, groin, feet, face, neck, or combinations thereof.

7. A method according to claim 1, wherein the ipratropium compound comprises a material selected from the group consisting of an ipratropium salt, an ipratropium analogue, an ipratropium derivative, an ipratropium prodrug, an ipratropium hydrate, an ipratropium isomer, or an ipratropium metabolite.

8. A method according to claim 1, wherein the ipratropium compound has the following formula: 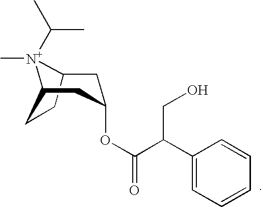
and may be optionally complexed with a pharmaceutically acceptable counter ion salt.

9. A method according to claim 1, wherein the composition comprises from about 0.25% (w/w) to about 25% (w/w) ipratropium compound.

10. A method according to claim 1, wherein the ipratropium compound comprises ipratropium bromide.

11. A method according to claim 1, wherein the topically administering does not include systemically distributing the ipratropium compound.

12. A method according to claim 1 further comprising identifying a subject susceptible to hyperhidrosis and/or excessive perspiration.

13. A method according to claim 1, wherein the composition is a dosage form selected from the group consisting of a liquid, a syrup, a paste, a cream, a lotion, an ointment, a gel, a stick, a roll-on, a spray, a foam, a mousse, and combinations thereof.

14. A method according to claim 13, wherein the liquid dosage form is selected from the group consisting of an aqueous solution, an alcoholic solution, and combinations thereof.

15. A method according to claim 1, wherein the composition further comprises a pharmaceutically acceptable excipient.

16. A method for ameliorating, preventing, and/or treating hyperhidrosis and/or excessive perspiration in a subject, the method comprising:
administering to the subject a dosage form comprising an effective amount of a pharmaceutically acceptable ipratropium compound, wherein hyperhidrosis and/or excessive perspiration is ameliorated, prevented, and/or treated and the dosage form is selected from the group consisting of a syrup, a paste, a cream, a lotion, an ointment, a gel, a stick, a roll-on, a spray, and combinations thereof.

17. A method according to claim 16, wherein the subject is a human and topically administering to the human further comprises topically administering to the subject's face, neck, hand, axillae, groin, feet, or combinations thereof.

18. A method according to claim 16, wherein the ipratropium compound comprises a material selected from the group consisting of an ipratropium salt, an ipratropium analogue, an ipratropium derivative, an ipratropium prodrug, an ipratropium hydrate, an ipratropium isomer, or an ipratropium metabolite.

19. A method for reducing perspiration in a human, the method comprising:
topically administering to the human a composition comprising an effective amount of a physiologically acceptable ipratropium compound, wherein perspiration is reduced.

20. A method according to claim 19, wherein the topically administering further comprises contacting the human with a medicated article comprising the composition comprising an effective amount of a pharmaceutically acceptable ipratropium compound.
